(19) United States
(12) Patent Application Publication
(10) Pub. No.: US 2013/0190500 A1
(43) Pub. Date: Jul. 25, 2013

(54) PROCESS TO PREPARE 6-CHLORO-3-AMINO-2-(2-HYDROXYPROPYL)-1-AZANAPHTHALENE

(71) Applicant: Neuron Systems, Inc., Burlington, MA (US)

(72) Inventors: Jack Fitzgerald Greiner, South Plainfield, NJ (US); Thomas Elliott Jacks, South Plainfield, NJ (US); Andrew Steven Thompson, South Plainfield, NJ (US)

(73) Assignee: NEURON SYSTEMS, INC., Burlington, MA (US)

(21) Appl. No.: 13/709,802

(22) Filed: Dec. 10, 2012

Related U.S. Application Data
(60) Provisional application No. 61/569,358, filed on Dec. 12, 2011.

(51) Int. Cl. C07D 215/38; C07C 221/00; C07D 215/48

(52) U.S. Cl. CPC C07D 215/38; C07D 215/48; C07C 221/00
USPC 546/159; 564/417

(57) ABSTRACT

This invention relates to a process for the preparation of 6-chloro-3-amino-2-(2-hydroxypropyl)-1-azanapthalene. It comprises treating 6-chloro-3-pyridylium-2-ethoxycarbonyl-1-azanaphthanlene bromide with morpholine at about 80° C., followed by treating the product thereof with methylmagnesium chloride.

### CROSS REFERENCE TO RELATED APPLICATIONS

This application claims benefit of U.S. Provisional Application Ser. No. 61/569,358, filed on Dec. 12, 2011. The contents of the prior application are incorporated herein by reference.

### BACKGROUND

Compound A (6-chloro-3-amino-2-(2-hydroxypropyl)-1-azanaphthalene) is in early stage clinical trials for the treatment of macular degeneration.

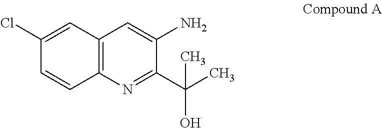 [SMILES: CC(C)(O)c1nc2ccc(Cl)cc2cc1N]

Although compound A can be readily prepared by those skilled in the art, more efficient synthetic routes are necessary for the production on a commercial scale. A highly efficient route to compound A is disclosed herein.

### SUMMARY

This invention relates to a process for the preparation of 6-chloro-3-amino-2-ethoxycarbonyl-1-azanaphthalene, which comprises treating 6-chloro-3-pyridylium-2-ethoxycarbonyl-1-azanaphthanlene bromide with morpholine at about 80° C. This process may further comprise treating ethyl pyridyliumpyruvate halide with 2-amino-5-chloro-benzaldehyde and pyridine at 80° C. under an inert atmosphere to yield 6-chloro-3-pyridylium-2-ethoxycarbonyl-1-azanapthalene halide mentioned above.

This invention also relates to a process for the preparation of 6-chloro-3-amino-2-(2-hydroxypropyl)-1-azanapthalene, which comprises the process for making 6-chloro-3-amino-2-ethoxycarbonyl-1-azanaphthalene described above, followed by treating the product thereof with methylmagnesium chloride.

In addition, this invention relates to a process for the preparation of 2-amino-5-chloro-benzalehyde, which comprises treating 5-chloro-2-nitrobenzaldehyde with hydrogen and 3% sulfided platinum.

### DETAILED DESCRIPTION OF THE INVENTION

The overall synthetic route to Compound A is shown in scheme 1 below.

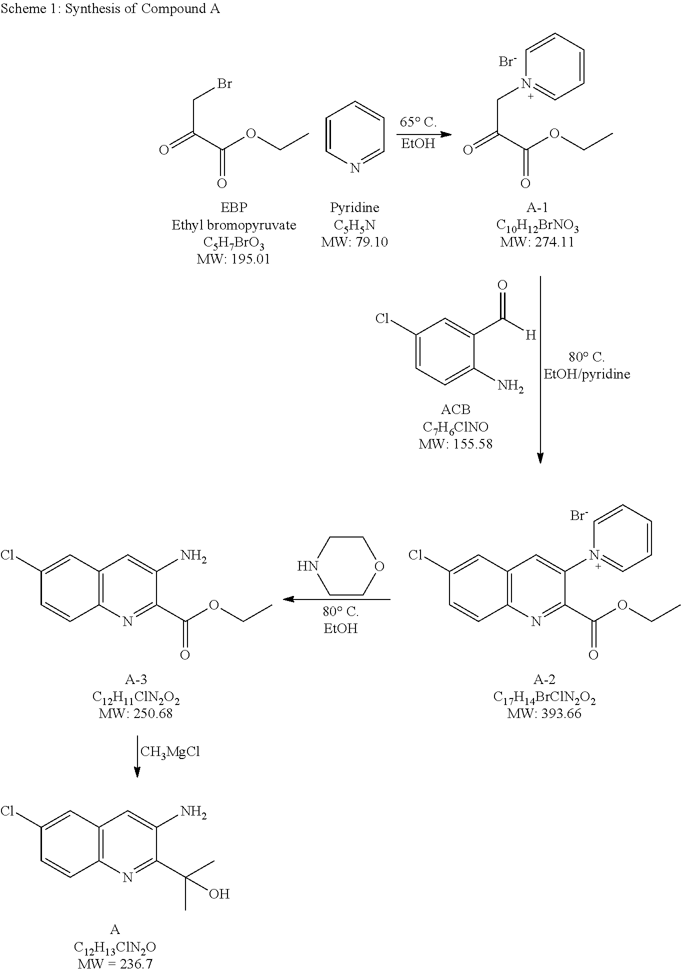

### EXAMPLE 1

### Preparation of A-1

To a 2 L round bottom flask was charged ethanol (220 mL), and pyridine (31 g, 392 mmol) and the resulting solution stirred at a moderate rate of agitation under nitrogen. To this solution was added ethyl bromopyruvate (76.6 g, 354 mmol) in a slow, steady stream. The reaction mixture was allowed to stir at 65±5° C. for 2 hours.

### EXAMPLE 2

### Preparation of A-2

Upon completion of the 2 hour stir time in example 1, the reaction mixture was slowly cooled to 18-22° C. The flask was vacuum-purged three times at which time 2-amino-5-chloro-benzaldehyde (ACB) (50.0 g, 321 mmol) was added directly to the reaction flask as a solid using a long plastic funnel. Pyridine (64.0 g, 809 mmol) was added followed by an EtOH rinse (10 mL) and the reaction mixture was heated at 80±3° C. under nitrogen for about 16 hours (overnight) at which time HPLC analysis indicated that the reaction was effectively complete.

### EXAMPLE 3

### Preparation of A-3

The reaction mixture from example 2 was cooled to about 70° C. and morpholine (76.0 g, 873 mmol)) was added to the 2 L reaction flask using an addition funnel. The reaction mixture was heated at 80±2° C. for about 2.5 hours at which time the reaction was considered complete by HPLC analysis (area % of A-3 stops increasing). The reaction mixture was cooled to 10-15° C. for the quench, work up, and isolation.

### EXAMPLE 4

### Isolation of A-3

To the 2 L reaction flask was charged water (600 g) using the addition funnel over 30-60 minutes, keeping the temperature below 15° C. by adjusting the rate of addition and using a cooling bath. The reaction mixture was stirred for an additional 45 minutes at 10-15° C. then the crude A-3 isolated by filtration using a Buchner funnel. The cake was washed with water (100 mL×4) each time allowing the water to percolate through the cake before applying a vacuum. The cake was air dried to provide crude A-3 as a nearly dry brown solid. The cake was returned to the 2 L reaction flask and heptane (350 mL) and EtOH (170 mL) were added and the mixture heated to 70±3° C. for 30-60 minutes. The slurry was cooled to 0-5° C. and isolated by filtration under vacuum. The A-3 was dried in a vacuum drying oven under vacuum and 35±3° C. overnight (16-18 hours) to provide A-3 as a dark green solid.

### EXAMPLE 5

### Preparation of Compound A

To a 2 L round bottom flask was charged methylmagnesium chloride (200 mL of 3.0 M solution in THF, 600 mmol). The solution was cooled to 0-5° C. using an ice bath.

A 500 mL flask (magnetic stirring) was charged with 22.8 grams A-3 from example 4 and THF (365 mL), stirred to dissolve then transferred to an addition funnel on the 2 L Reaction Flask. The A-3 solution was added drop-wise to the reaction flask over 5.75 hours, keeping the temperature of the reaction flask between 0-5° C. throughout the addition. At the end of the addition the contents of the flask were stirred for an additional 15 minutes at 0-5° C. then the cooling bath was removed and the reaction was allowed to stir overnight at ambient temperature.

The flask was cooled in an ice bath and the reaction mixture was carefully quenched by adding EtOH (39.5 g, 857 mmol) drop-wise to the reaction mixture, keeping the temperature of the reaction mixture below 15° C. during the course of the addition. An aqueous solution of NH₄Cl (84.7 g NH₄Cl in 415 mL water) was then carefully added and the mixture stirred under moderate agitation for about 30 minutes then transferred to a separatory funnel to allow the layers to separate. Solids were present in the aqueous phase so HOAc (12.5 g) was added and the contents swirled gently to obtain a nearly homogeneous lower aqueous phase. The lower aqueous layer was transferred back to the 2 L reaction flask and stirred under moderate agitation with 2-methylTHF (50 mL) for about 15 minutes. The original upper organic layer was reduced in volume to approximately 40 mL using a rotary evaporator at ≦40° C. and vacuum as needed. The phases in the separatory funnel were separated and the upper 2-MeTHF phase combined with the product residue, transferred to a 500 mL flask and vacuum distilled to an approximate volume of 25 mL. To this residue was added 2-MeTHF (50 mL) and distilled to an approximate volume of 50 mL. The crude compound A solution was diluted with 2-MeTHF (125 mL), cooled to 5-10° C. and 2M H₂SO₄ (aq) (250 mL) was slowly added and the mixture stirred for 30 minutes as the temperature was allowed to return to ambient. Heptane (40 mL) was charged and the reaction mixture stirred for an additional 15 minutes then transferred to a separatory funnel and the layers were allowed to separate. The lower aqueous product layer was extracted with additional heptane (35 mL) then the lower aqueous phase was transferred to a 1 L reaction flask equipped with a mechanical stirrer and the mixture was cooled to 5-10° C. The combined organic layers were discarded. A solution of 25% NaOH(aq) was prepared (NaOH, 47 g, water, 200 mL) and slowly added to the 1 L reaction flask to bring the pH to a range of 6.5-8.5.

EtOAc (250 mL) was added and the mixture was stirred overnight. The mixture was transferred to a separatory funnel and the lower phase discarded. The upper organic layer was washed with brine (25 mL) then the upper organic product layer was reduced in volume on a rotary evaporator to obtain a obtain the crude compound A as a dark oil that solidified within a few minutes. The crude compound A was dissolved in EtOAc (20 mL) and filtered through a plug of silica gel (23 g) eluting with 3/1 heptane/EtOAc until all compound A was eluted (approximately 420 mL required) to remove most of the dark color of compound A. The solvent was removed in vacuo to provide 14.7 g of compound A as a tan solid. Compound A was taken up in EtOAc (25 mL) and eluted through a column of silica gel (72 g) using a mobile phase gradient of 7/1 heptane/EtOAc to 3/1heptane/EtOAc (1400 mL total) . The solvent fractions containing compound A were stripped, compound A diluted with EtOAc (120 mL) and stirred in a flask with Darco G-60 decolorizing carbon (4.0 g) for about 1 hour. The mixture was filtered through celite using a firtted funnel, rinsing the cake with EtOAc (3×15 mL). The combined filtrates were stripped on a rotary evaporator and compound A dissolved in heptane (160 mL)/EtOAc(16 mL) at 76° C. The homogeneous solution was slowly cooled to 0-5° C., held for 2 hours then compound A was isolated by filtration. After drying in a vacuum oven for 5 hours at 35° C. under best vacuum, compound A was obtained as a white solid.

HPLC purity: 100% (AUC)
HPLC (using standard conditions):
A-2: 7.2 minutes
A-3: 11.6 minutes

### EXAMPLE 6

### Preparation of ACB

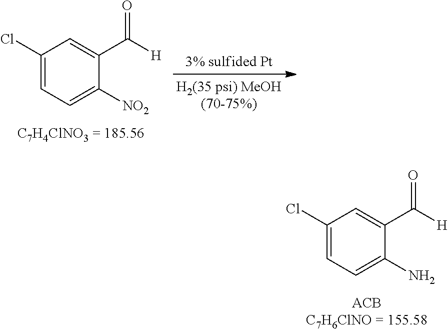 [SMILES: O=Cc1cc(Cl)ccc1[N+](=O)[O-] ; Nc1ccc(Cl)cc1C=O]

After a N₂ atmosphere had been established and a slight stream of N₂ was flowing through the vessel, platinum, sulfided, 5 wt % on carbon, reduced, dry (9.04 g, 3.0 wt % vs the nitro substrate) was added to a 5 L heavy walled pressure vessel equipped with a large magnetic stir-bar and a thermocouple. MeOH (1.50 L), 5-chloro-2-nitrobenzaldehyde (302.1 g, 1.63 mol), further MeOH (1.50 L) and Na₂CO₃ (2.42 g, 22.8 mmol, 0.014 equiv) were added. The flask was sealed and stirring was initiated at 450 rpm. The solution was evacuated and repressurized with N₂ (35 psi), 2×. The flask was evacuated and repressurized with H₂ to 35 psi. The temperature of the solution reached 30° C. w/in 20 min. The solution was then cooled with a water bath. Ice was added to the water bath to maintain a temperature below 35° C. Every 2 h, the reaction was monitored by evacuating and repressurizing with N₂ (5 psi), 2× prior to opening. The progress of the reaction could be followed by TLC: 5-Chloro-2-nitrobenzaldehyde (Rf=0.60, CH₂Cl₂, UV) and the intermediates (Rf=0.51, CH₂Cl₂, UV and Rf=0.14, CH₂Cl₂, UV) were consumed to give ACB (Rf=0.43, CH₂Cl₂, UV). At 5 h, the reaction had gone to 98% completion (GC),and was considered complete. To a 3 L medium fritted funnel was added celite (ca. 80 g). This was settled with MeOH (ca. 200 mL) and pulled dry with vacuum. The reduced solution was transferred via cannula into the funnel while gentle vacuum was used to pull the solution through the celite plug. This was chased with MeOH (150 mL 4×). The solution was transferred to a 5 L three-necked round-bottom flask. At 30° C. on a rotavap, solvent (ca. 2 L) was removed under reduced pressure. An N₂ blanket was applied. The solution was transferred to a 5 L four-necked round-bottomed flask equipped with mechanical stirring and an addition funnel. Water (2.5 L) was added dropwise into the vigorously stirring solution over 4 h. The slurry was filtered with a minimal amount of vacuum. The collected solid was washed with water (1.5 L 2×), iPA (160 mL) then hexanes (450 mL 2×). The collected solid (a canary yellow, granular solid) was transferred to a 150×75 recrystallizing dish. The solid was then dried under reduced pressure (26-28 in Hg) at 40° C. overnight in a vacuum-oven. ACB (>99A % by HPLC) was stored under a N₂ atmosphere at 5° C.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims. It will be understood by those skilled in the art that various changes in form and details may be made therein without departing from the scope of the invention encompassed by the appended claims.

1. A process for the preparation of 6-chloro-3-amino-2-ethoxycarbonyl-1-azanaphthalene comprising treating 6-chloro-3-pyridylium-2-ethoxycarbonyl-1-azanaphthanlene bromide with morpholine at about 80° C.

2. The process of claim 1 further comprising treating ethyl pyridyliumpyruvate halide with 2-amino-5-chloro-benzaldehyde and pyridine at 80° C. under an inert atmosphere to yield 6-chloro-3-pyridylium-2-ethoxycarbonyl-1-azanapthalene halide.

3. A process for the preparation of 6-chloro-3-amino-2-(2-hydroxypropyl)-1-azanapthalene comprising the process of claim 2 followed by treating the product thereof with methylmagnesium chloride.

4. A process for the preparation of 2-amino-5-chloro-benzalehyde comprising treating 5-chloro-2-nitrobenzaldehyde with hydrogen and 3% sulfided platinum.
